(19) United States
(12) Patent Application Publication
(10) Pub. No.: US 2017/0252525 A1
(43) Pub. Date: Sep. 7, 2017

(54) RESPIRATORY ASSISTANCE APPARATUS USABLE IN CARDIOPULMONARY RESUSCITATION

(71) Applicant: Air Liquide Medical Systems, Antony (FR)

(72) Inventors: Maxence FOURNIER, Paris (FR); Eric JACQUOT, Antony (FR); Mickaël LIBARDI, Le Kremlin Bicetre (FR)

(21) Appl. No.: 15/450,113

(22) Filed: Mar. 6, 2017

(30) Foreign Application Priority Data
Mar. 7, 2016 (FR) 1651885

(51) Int. Cl. A61M 16/00

(52) U.S. Cl. CPC A61M 16/0069; A61M 2016/003; A61M 2205/502; A61M 2205/3331; A61M 2016/0027

(57) ABSTRACT

The invention relates to a respiratory assistance apparatus (1) usable by a first responder, such as a doctor from the emergency ambulance service, a fire fighter, a nurse or the like, when this first responder is performing cardiac massage on an individual (20) in cardiopulmonary arrest, in order to ventilate the said individual while he or she is being subjected to chest compressions. According to the invention, the respiratory assistance apparatus (1) comprises signal processing means designed to supply the display means with a corrected frequency value (F_{c}) corresponding to the frequency value (F) determined immediately before the start of a time of duration (Dh, Db) considered, when the signal processing means do not detect a chest compression (CC) for part of the said duration (Dh, Db) considered.

### CROSS REFERENCE TO RELATED APPLICATIONS

This application claims the benefit of priority under 35 U.S.C. §119 (a) and (b) to French Patent Application No. 1651885 filed Mar. 7, 2016, the entire contents of which are incorporated herein by reference.

### BACKGROUND

The invention relates to an artificial ventilation device or apparatus, also referred to as a respiratory or ventilation assistance apparatus, and more particularly to a respiratory assistance apparatus usable by a first responder, such as a doctor of the emergency ambulance service, a firefighter, a nurse or the like, when this first responder is performing cardiac massage on an individual, namely a patient, in cardiopulmonary arrest, in order to ventilate the said individual while he or she is being subjected to chest compressions.

Certain types of respiratory assistance apparatus or ventilator are fitted to emergency response vehicles which notably respond to road traffic accidents or when emergency medical care is required, such as in the event of heart attacks.

Thus, in the event of a heart attack, it is of prime importance to combine mechanical ventilation with cardiac massage performed on the individual in cardiopulmonary arrest, which means to say to perform cardiopulmonary resuscitation (CPR).

Numerous studies have revealed that the frequency of cardiac massage is a key factor in determining its efficiency. There is a consensus that sets the minimum frequency for cardiac massage at 100 compressions per minute. This minimum frequency is notably recommended by the American Heart Association in “Guidelines for Cardiopulmonary Resuscitation (CPR) and Emergency Cardiovascular Care (ECC)”; 2010.

Now, in practice, it has been found that the cardiac massage performed is often too slow, as attested to in “Chest Compression Rates During Cardiopulmonary Resuscitation Are Suboptimal: A Prospective Study During In-Hospital Cardiac Arrest”; American Heart Association; Abella et al. 2005

Consequently, it would seem essential to provide the first responder or the like with information relating to the frequency of the cardiac massage he or she is performing so as to allow him/her to adjust his/her cardiac massage, which means to say to increase chest compressions (CCs) if these are insufficient to achieve the recommended minimal frequency.

Document U.S. Pat. No. 5,496,257 describes a device to be placed on the patient's thorax during cardiac massage and which is capable of measuring the frequency of the CCs and of supplying information relating to this frequency, and document U.S. Pat. No. 6,390,996 describes a similar device which is fixed around the first responder's wrist.

One major disadvantage with such devices is that they add additional devices to all of the equipment that the first responder requires, thus delaying the start of cardiac massage during CPR, when speed is a key factor in determining the survival of the patient.

In addition, the first device makes the intervention more complicated because it has to be positioned between the hands of the first responder and the thorax of the patient, whereas the second device mentioned does not allow two first responders to take over from one another easily during massage unless each is provided with such a device.

One major problem is therefore that of being able to provide the first responder with information about the frequency of the CCs without having to resort to an additional device so as not to make the first responder's task any more complicated.

In an attempt to address this, document FR-A-3000893 teaches a respiratory assistance apparatus comprising a gas source, such as motorized turbine, for intermittently delivering a respiratory gas to a ventilation circuit supplying a patient who is being subjected to CPR; measurement means able to measure and deliver a signal representative of a parameter representative of the flow of gas, such as flow rate, pressure, etc.; signal processing means for analysing the said signal and from it deducing whether or not cardiac massage is being performed, namely whether CCs are present or absent, and of determining the frequency thereof; and a display screen for displaying the frequency of the CCs.

Now, in practice, it has been found that, with this type of device, the existence of CCs is not always correctly detected during the moments in time for which the gas is being delivered to the patient, thereby falsifying the calculation of the frequency of the said CCs and causing the first responder or other member of care personnel to be delivered erroneous information.

The problem that arises is therefore that of proposing a respiratory assistance apparatus that is able to solve all or some of the aforementioned problems and disadvantages, particularly a respiratory assistance apparatus that makes it possible to supply the first responder with reliable information regarding the frequency of the CCs, even when the existence of such CCs is not correctly detected by the apparatus, and to do so without having to resort to an additional device so as not to make the first responder's task any more complicated.

### SUMMARY

The solution is therefore a respiratory assistance apparatus comprising:
a gas source able to deliver a flow of respiratory gas in a ventilation circuit intended to be connected to a patient, the flow of respiratory gas being delivered by the said gas source in such a way as to generate and maintain several gas pressure levels (Ph, Pb) for times of preset duration (Dh, Db),
measurement means able and designed to measure at least one flow parameter representative of the gas flow and chosen from the pressure of the gas and the gas flow rate, and to deliver at least one flow signal representative of the said flow parameter,
signal processing means designed and able to, i.e. configured to:
   i) analyse and deduce from the said flow signal, the existence or absence of chest compressions performed on the said patient,
   ii) determine the value of the frequency (F) of the said chest compressions in the event that chest compressions exist,
   iii) supply the said frequency value (F) to display means,
and display means allowing information useful to the first responder, in particular the value of the frequency (F) of the chest compressions or any other useful item of data or information, to be displayed,

characterized in that the signal processing means are designed to supply the display means with a corrected frequency value (F_{c}) corresponding to the frequency value (F) determined immediately before the start of a time of duration (Dh, Db) considered, when the signal processing means do not detect a chest compression for part of the said duration (Dh, Db) considered.

In other words, the gas source is configured to deliver a flow of respiratory gas at several gas pressure levels (Ph, Pb) for times of preset duration (Dh, Db) and the apparatus of the invention has an operation based on taking into consideration chest compressions (CCs) that are virtual, which means to say CCs that are not detected during the durations (Dh, Db) in which the gas pressure levels (Ph, Db) are being maintained even though such compressions are in fact being performed, and on displaying an estimate of the frequency of these undetected CCs. Specifically, when the mechanical ventilation gives rise to significant variations in the pressure and flow rate signals, actual CCs may be obscured and the calculated frequency may be erroneous.

According to the invention, virtual CCs are therefore added if no actual CC is detected, so as to compensate for the loss of information caused by interference between the cardiac massage and the mechanical ventilation, namely the supply of gas by the gas source and the maintaining of the gas pressure levels, typically a motorized microblower, with which the ventilation apparats is equipped.

Stated differently, a corrected CC frequency (F_{c}) is obtained by estimating virtual CCs, which amounts to taking into consideration and displaying not an actual frequency (F) that could be erroneous, but a corrected or virtual frequency (F_{c}) corresponding to the frequency determined by the apparatus, and preferably stored in memory, just before the start of each period of time during which a gas pressure level is maintained.

By virtue of the invention, the calculation and display of the corrected frequency of the CCs using estimates of virtual CCs if the apparatus does not detect any cardiac massage even though massage is taking place allows better guidance for the first responder performing the cardiac massage on the ventilated patient and allows him or her to make sure that the minimum frequency of 100 compressions per minute is indeed being respected or, if need be, to correct his or her way of applying the massage in order to obtain this minimum frequency.

The apparatus of the invention may, as the case may be, comprise one or more of the following technical features:
the display means are suitable for displaying the said corrected frequency value (F_{c}) for at least part of the duration (Dh, Db) considered.
The display means are designed and/or configured to display the frequency directly or indirectly. Thus, they may display one (or more) frequency value directly, or indirectly via a graphical representation of the frequency value, for example in the form of a bar chart or the like, or via indication(s) of the “too fast”, “too slow”, “speed up massage”, “slow down massage” type or any other suitable illustrative symbol, etc. This provides the first responder with very useful information as to the effectiveness of the cardiac massage he or she is in the process of performing.
The display means are designed and/or configured to display, directly or indirectly, information other than information associated with the frequency.
The display means comprise a display screen or the like, namely a display or a digital screen, preferably a touchscreen.
The gas source delivers the flow of respiratory gas and maintains the gas pressure levels (Ph, Pb) of the said flow, for times (Dh, Db) of preset duration such that:
a) the gas is kept at a high pressure (Ph) for a first duration (Dh), and
b) the gas is kept at a low pressure (Pb) for a second duration (Db), where: 0<Pb<Ph, the second duration (Db) following on from the first duration (Dh).
The gas source maintains the gas pressure levels (Ph, Pb) of the said flow for preset durations (Dh, Db) that alternate cyclically.
The first duration (Dh) of the first phase is less than 4 seconds.
The second duration (Db) of the second phase is less than 12 seconds.
The total duration of each cycle (Dh+Db) is less than 15 seconds, typically less than 8 seconds, typically of the order of 5 seconds.
The high pressure (Ph) delivered by the gas source is comprised between 100 and 400 mmH₂O, preferably between 150 and 300 mmH₂O, typically of the order of 200 mmH₂O.
The low pressure (Pb) delivered by the gas source is comprised between 20 and 90 mmH₂O, preferably between 30 and 70 mmH₂O, typically of the order of 40 mmH₂O.
It comprises control means operating the gas source in such a way as to deliver the flow of gas and to generate the pressure levels (Ph, Pb) for the preset durations (Dh, Db).
The control means operate the gas source in such a way as to deliver the flow of gas at high pressure (Ph) and/or at low pressure (Pb).
The gas source is a motorized microblower.
The measurement means comprise a flow sensor or a pressure sensor.
The signal processing means are configured to supply the display means with the corrected frequency value (F_{c}) when the signal processing means do not detect a chest compression for a duration equal to 10 to 60% of the duration (Dh, Db) considered.
The signal processing means comprise at least one microprocessor implementing at least one algorithm.
It comprises filtering means configured to filter the flow signal representative of the flow parameter coming from the measurement means and to supply a filtered flow signal to the signal processing means.
The signal processing means are configured to analyse a filtered flow signal and from it deduce the existence or absence of chest compressions (CCs) performed on a patient.
The measurement means make it possible to measure at least one flow parameter within the said ventilation circuit.
The signal processing means are designed, notably programmed, to compare the said at least one flow signal with at least one threshold value representative of cardiac massage being performed, particularly a threshold value that is preset and/or stored in memory by the apparatus, particularly in information memory storage means.
It further comprises duration measurement means making it possible, namely able and designed to, determine the duration of time for which cardiac massage is performed.
The duration measurement means comprise a timer incorporated into a processor.
The display means are designed also to display the duration of the cardiac massage.
It comprises memory storage means designed and able to store data, preferably one (or more) memory card, for example a flash memory.
The signal processing means are configured to determine the value of the frequency (F) of the chest compressions by calculating, over a given duration, the ratio between the number of chest compressions and the said given duration; the said ratio obtained then corresponds to the frequency F of the CCs. For preference, the duration is comprised between 1 and 15 seconds, preferably between 2 and 10 seconds, typically of the order of 4 to 8 seconds, for example around 6 seconds.
It comprises means of counting the chest compressions Cs detected over a given period of time, for example the counting means comprise a CC counter that is reset at the start of the said time period, and incremented each time a new CC is detected during the said time period.
The counting means cooperate with the memory storage means by supplying the said memory storage means with a number of CCs detected over a given period.
It comprises electrical current supply means, preferably one or more batteries, preferably at least one rechargeable battery. The electrical current supply means power the various components of the apparatus, notably the display screen, the information processing means, the measurement means, etc.
It comprises a means of switching the operation of the apparatus on and/or off, for example a key, a button or the like of the on/off type.

According to another aspect, the invention also relates to a method for ventilating an individual, namely a patient, in cardiopulmonary arrest, comprising the steps of:

i) performing cardiac massage on an individual in cardiopulmonary arrest, by performing chest compressions on the said individual, and

ii) ventilating the individual in cardiopulmonary arrest, preferably simultaneously with step i), by administering to the said individual a flow of respiratory gas coming from a respiratory assistance apparatus according to the present invention, the flow of respiratory gas being delivered by the gas source of the respiratory assistance apparatus according to the present invention in such a way as to generate and maintain several gas pressure levels (Ph, Pb) for times of preset duration (Dh, Db),

iii) using the respiratory assistance apparatus according to the present invention to measure at least one flow parameter representative of the gas flow and chosen from the gas pressure and the gas flow rate, and for delivering at least one flow signal representative of the said flow parameter,

iv) processing the said flow signal within the respiratory assistance apparatus according to the present invention in order to deduce therefrom the existence or absence of chest compressions performed on the said patient, and determine the value of the frequency (F) of the said chest compressions if such chest compressions exist, and

v) displaying, preferably on display means, such as a display screen or the like, of the respiratory assistance apparatus according to the invention, a corrected frequency value (F_{c}) corresponding to the frequency value (F) determined immediately before the start of a duration (Dh, Db) considered, when the signal processing means do not detect a chest compression for part of the said duration (Dh, Db) considered.

Depending on the circumstances, the frequency may be displayed directly or indirectly. Thus, it is possible to display one (or more) frequency values directly, or indirectly via a graphical representation of the frequency value, for example in the form of a bar chart, or the like, or via indication(s) of the “too fast”, “too slow”, “speed up massage”, “slow down massage” type or any other suitable illustrative symbol, etc. That provides the first responder with very useful information as to the effectiveness of the cardiac massage he or she is in the processor of performing.

The method of the invention may further comprise all or some of the other features described in the present description.

### BRIEF DESCRIPTION OF THE DRAWINGS

The present invention will now be described in greater detail with reference to the attached figures among which:

FIG. 1 depicts one embodiment of a respiratory assistance apparatus according to the present invention;

FIG. 2 is an example of the pressure parameter recorded by a respiratory assistance apparatus in the absence of cardiac massage;

FIG. 3 illustrates the operation of one example of an algorithm used by the control means of the ventilator of FIG. 1 according to the present invention.

### DESCRIPTION OF THE PREFERRED EMBODIMENTS

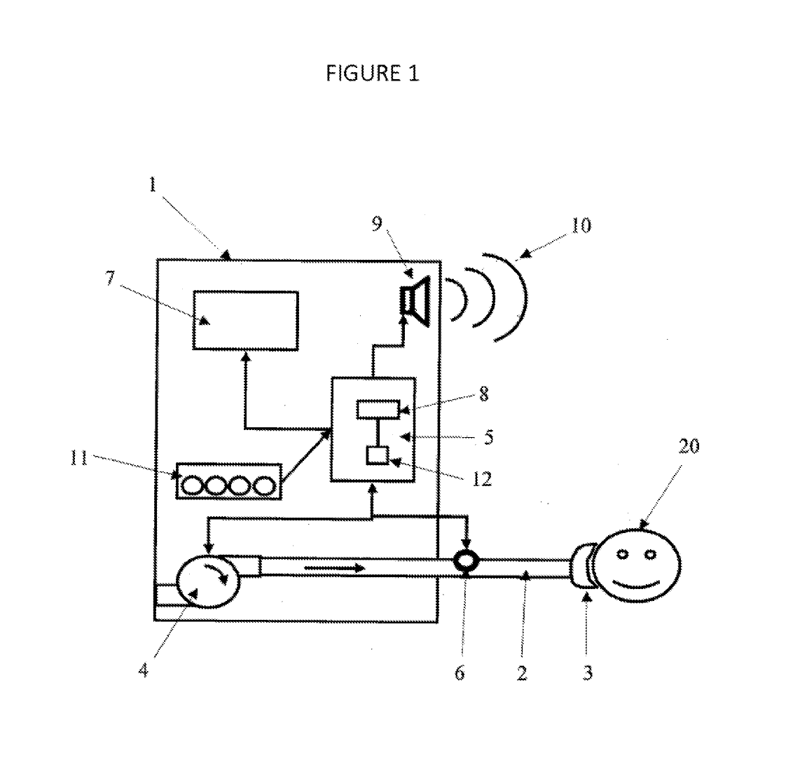

FIG. 1 schematically depicts one embodiment of a ventilation assistance apparatus or medical ventilator 1 according to the present invention.

The ventilator 1 comprises a motorized microblower 4 delivering a flow of respiratory assistance gas, typically a flow of air or of oxygen-enriched air, in a ventilation circuit 2, also referred to as a patient circuit, comprising one or more gas passages or lines fluidically connecting the ventilator 1 to the airways of a patient 20, via a patient interface 3, such as a respiratory mask or an intubation tube.

The gas source 4, typically a motorized microblower, also referred to as a turbine or compressor, is controlled by control means to deliver the flow of respiratory gas and to generate pressure levels (Ph, Pb) intermittently, for preset durations (Dh, Db).

The motorized microblower 4 is equipped with an electric motor driving a vaned wheel used to create the flow of gas which is then delivered upstream of the patient circuit formed by one or more gas passages, such as pipes or the like, used to convey the gas as far as the patient 20.

The gas source 4 delivers the flow of respiratory gas and generates pressure levels (Ph, Pb) for times of preset duration (Dh, Db).

In general, the first duration (Dh) of the first phase is less than 4 seconds, and/or the second duration (Db) of the second phase is less than 12 seconds. Furthermore, the total duration of each cycle (Dh+Db) is less than 15 seconds, typically less than 8 seconds, typically of the order of 5 seconds.

During the first phase of first duration (Dh) of each cycle, the gas is supplied at a first non-zero pressure value or “high pressure Ph”, whereas during the second phase of second duration (Db), which follows on from the first phase (Dh), the gas is delivered at a second non-zero pressure value or “low pressure Pb”, the high pressure Ph being higher than the low pressure Pb, which means to say that 0<Pb<Ph.

For preference, the high pressure (Ph) delivered by the gas source is comprised between 100 and 400 mmH₂O, preferably between 150 and 300 mmH₂O, typically of the order of 200 mmH₂O.

Similarly, the low pressure (Pb) delivered by the gas source is preferably comprised between 20 and 90 mmH₂O, preferably between 30 and 70 mmH₂O, typically of the order of 40 mmH₂O.

The control means then control the gas source in such a way to deliver the gas flow and to generate the pressure levels (Ph, Pb) for the times of preset duration (Dh, Db).

According to another embodiment (not depicted), the microblower 4 may be replaced by another gas source, such as a valve, connected to a gas pipeline, particularly a gas pipeline ending in a wall outlet arranged on a wall of a hospital building and supplied with respiratory gas, such as air or oxygen, at a pressure of a few bar absolute, typically of the order of around 4 bar abs.

Moreover, in the ventilator 1 of FIG. 1, there are also provided measurement means 6 making it possible to measure at least one flow parameter representative of the gas flow, typically the pressure or the flow rate of gas blown in by the respirator, and to deliver at least one flow signal representative of the said at least one flow parameter measured.

For example, the gas flow parameter is the pressure of the gas in the ventilation circuit 2 and the measurement means 6 comprise a pressure sensor the pressure tapping of which is arranged in the said circuit 2 in such a way as to measure there the pressure prevailing therein.

In the embodiment of FIG. 1, the pressure tapping 6 is arranged away from the ventilator. However, according to the embodiment considered, it may just as well be situated away from as in the ventilator 1.

Once the gas flow parameter measurement (or measurements) has (or have) been taken, the corresponding flow signal, for example a flow rate or pressure signal, is analysed by signal processing means 5 which can then detect chest compressions or CCs therein and therefore from that deduce that the patient 20 is in the process of receiving cardiac massage, and hence from that also determine the frequency F of the said CCs. For preference, the signal is filtered as explained hereinbelow.

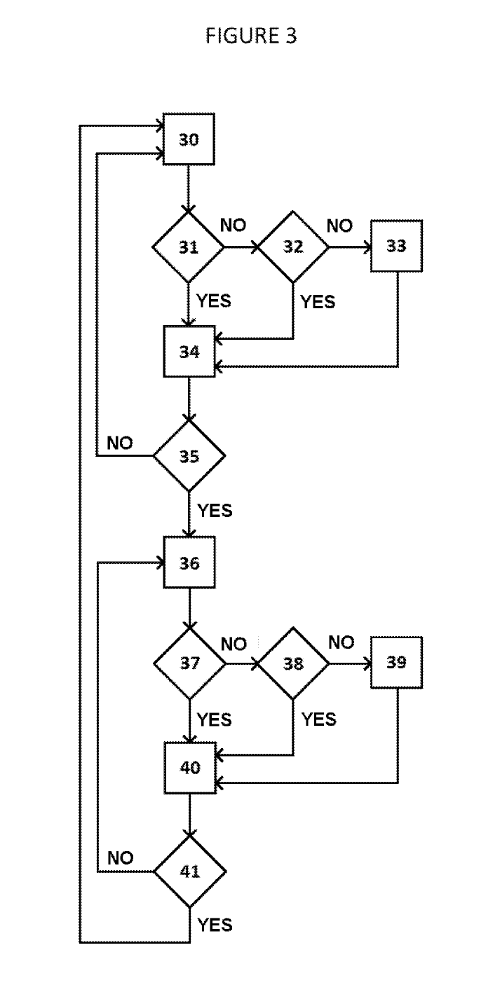

The signal processing means 5 comprise for example a programmable microprocessor 8, notably with a processing algorithm, as explained hereinafter, notably with reference to FIG. 3.

More specifically, the signal processing means 5 are able and designed to, which means to say configured to, compare the flow signal with one or more threshold values representative of cardiac massage being performed, which means to say corresponding to CCs in the process of being performed on the patient 20. These threshold values are recorded by memory storage means 12 comprising a storage memory, for example a flash memory. These threshold values may be numerical values, tables of values, curves, etc.

The apparatus further comprises filtering means, for example a sliding mean calculated across the latest values of a signal that are held in memory. Specifically, the chest compressions are advantageously brought to light by a filtering of the pressure and/or flow rate signals in the form of a subtraction of sliding means. Thus, for one and the same, pressure or flow rate, signal, it is possible for example to calculate a “large” sliding mean over a duration of 10 to 100 ms, typically 25 ms, and a “large” sliding mean over a duration of 50 to 300 ms, typically 150 ms. Subtracting the big sliding mean from the small sliding mean for example thus makes it possible to reveal variations in the said signal, in the form of a filtered signal. The chest compressions Cs are then detected when this filtered signal exceeds threshold values.

In other words, according to the present invention, the operation of the apparatus is preferably based on a use of filtered flow signal values, particularly filtered pressure or flow rate values.

Once they have been detected, the CCs can then be counted by counting means included in the signal processing means 5 in order therefrom to detect a value for the frequency of the cardiac massage by calculating, over a certain duration, the ratio between the number of chest compressions and the said duration. For preference, the said duration is comprised between 1 and 15 seconds, preferably between 2 and 10 seconds, typically of the order of 4 to 8 seconds, for example around 6 seconds.

Once the frequency F of the cardiac massage has been estimated by the signal processing means 5, this value can be refined by compensating for the actual CCs that are concealed by the high variations in pressure and flow rate that occur at the start and end of the durations (Dh, Db) by introducing virtual CCs and displaying on a display screen 7 or the like of the apparatus 1, a corrected frequency value F_{c} that takes these concealed, which means to say undetected, CCs into consideration in order to determine the most appropriate frequency information to display to the first responder.

In other words, the signal processing means 5 are designed, which means to say configured, to supply the display means 7 with a corrected frequency value F_{c} corresponding to the frequency value F determined immediately before the start of a given duration (Dh, Db) corresponding to a duration during which the microblower 4 of the ventilator 1 is supplying respiratory gas and generating a pressure level (Ph, Pb) when the signal processing means 5 do not detect a chest compression during part of the said duration (Dh, Db) considered by processing of the filtered flow signal.

This corrected frequency value F_{c} is then displayed on the display screen 7 of the ventilator 1, in place of the actual frequency which is itself erroneous because of the absence of detection of certain CCs.

Thus, at the start of each duration (Dh, Db), for a certain time (Th, Tb) comprised within the said duration (Dh, Db), for example representing between 10% and 60% of the said duration (Dh, Db), typically 30% (in particular 0<Th<Dh and 0<Tb<Db), the apparatus 1 is allowed to record virtual chest compressions if no actual CC is detected when the period corresponding to the calculated frequency has elapsed.

This then avoids an underestimation of the frequency of the cardiac massage caused by an absence of detection.

Furthermore, if a virtual CC is recorded, the detection of a new actual CC during a predefined duration, for example of between 50 and 350 ms, typically of 120 ms, is prevented, so that this chest compression is not counted twice, once in the form of a virtual CC and once in the form of an actual CC. Specifically, when the cardiac massage is being performed manually in particular the frequency of the massage is not of perfect regulatory, and an actual CC may occur very slightly later than the calculated frequency might predict. If that happens, and if that actual CC has already been accounted for through a virtual CC, then the actual CC must no longer be acknowledged, because if it were, the frequency of the massage would be overestimated.

The apparatus 1 according to the present invention provides assistance to the first responder by giving him/her an item of frequency information for example the numerical value of the frequency, or indications of the “too fast”, “too slow”, etc. type, which will provide him/her with very useful information as to the effectiveness of the cardiac massage he/she is in the process of performing. This frequency information is displayed on display means 7 such as a screen or a display.

Moreover, duration measurement means, for example a timer incorporated into a processor 8, may also be provided for measuring the duration for which the cardiac massage is performed. This duration will then be transmitted to the display means 7 to display this duration of cardiac massage for the attention of the care staff thus instantly supplying them with another important piece of information.

The ventilator 1 may also comprise a function of the “metronome” type that will assist the care personnel is performing cardiac massage at a given rate.

The ventilator 1 further comprises a rigid external shell or casing, for example made of polymer, incorporating all or some of the aforementioned means and elements, particularly the microblower 4, the signal processing means 5, including the microprocessor(s), the memory storage means, the display means 7, at least part of the ventilation circuit 2, etc.

The ventilator 1 is supplied with electrical current from one or more batteries, which may or may not be rechargeable, from the electric power supply of the emergency response vehicle which it equips, or from the mains, therefore at a voltage that may be as high as around 230 V.

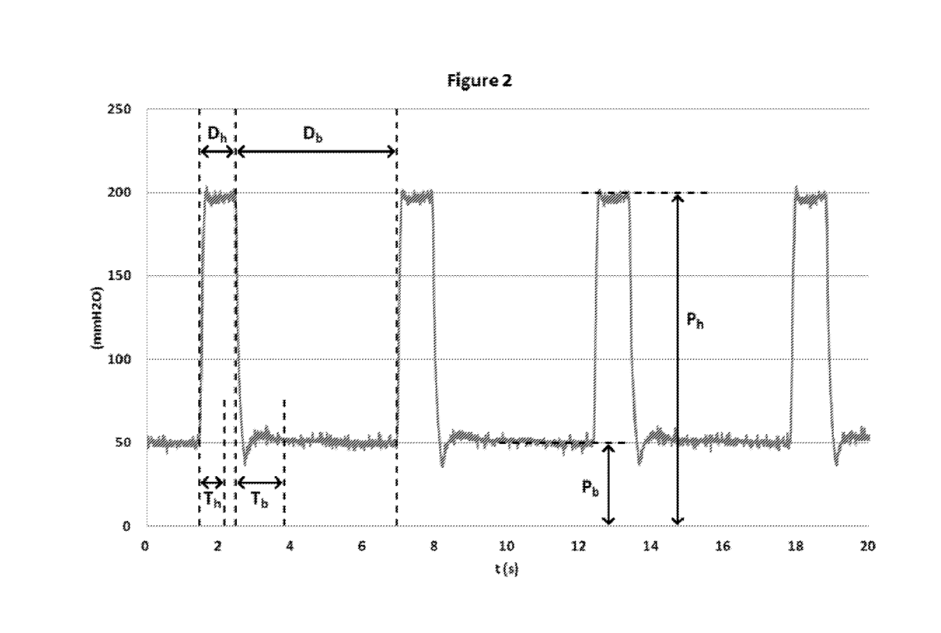

FIG. 2 is an example of a recording of a pressure parameter measured as a function of time, in the absence of cardiac massage. This recording was made using a Monnal T60 ventilation apparatus marketed by Air Liquide Medical Systems.

FIG. 2 schematically depicts the durations Dh and Db for which the respective gas pressure levels Ph and Pb are maintained, and the times Th and Tb respectively comprised within the said durations Dh and Db and during which the apparatus 1 is permitted to record virtual chest compressions if no actual CC is detected when the period corresponding to the calculated frequency has elapsed.

The flow diagram in FIG. 3 illustrates the operation of an algorithm that can be run within the signal processing means 5 of the ventilator 1 according to the invention.

As can be seen, the ventilator 1 first of all begins by applying the pressure level Ph (at 30). If the time elapsed since the start of the pressure level Ph is shorter than the time Th (at 31), the ventilator 1 checks whether actual CCs have been detected (at 32). If they have, these CCs are recorded directly for the frequency calculation (at 34). If not, virtual CCs are added (at 33) to be taken into consideration in the frequency calculation (at 34) and to compensate for the actual CCs concealed by the mechanical ventilation. If the time elapsed since the start of the pressure level Ph is longer than the time Th (at 31), only the actual CCs are recorded for the frequency calculation (at 34).

If the time elapsed since the start of the pressure level Ph is shorter than the duration Dh (at 35), the ventilator 1 continues to apply the pressure level Ph (at 30). If not, the ventilator applies the pressure level Pb (at 36). If the time elapsed since the start of the pressure level Pb is shorter than the time Tb (at 37), the ventilator 1 checks whether actual CCs have been detected (at 38). If they have, these CCs are recorded directly for calculating the frequency (at 40). If not, virtual CCs are added (at 39) to be taken into consideration in the frequency calculation (at 40) and to compensate for the actual CCs concealed by the mechanical ventilation. If the time that has elapsed since the start of the pressure level Pb is longer than the time Tb (at 37), only the actual CCs are recorded for the frequency calculation (at 40).

If the time that has elapsed since the start of the pressure level Pb is shorter than the duration Db (at 41), the ventilator 1 continues to apply the pressure level Pb (at 36). If not, the ventilator applies the pressure level Ph (at 30) and so on, cyclically.

Stated a different way, the permanent monitoring of the pressure, or flow rate, signals makes it possible, through conventional signal analysis, for example filtering or pattern recognition, to detect cardiac massage. Once massage has been detected, the frequency can be calculated by the same conventional signal processing techniques.

The duration since the start of the massage can also be displayed. Thus it can be timed by the processor.

The respiratory assistance apparatus according to the invention may be of portable type and provided with a carry handle or the like.

For mobility purposes, the said at least one gas source may be a turbine and/or a pressurized gas cylinder, typically fitted with a regulator.

The respiratory assistance apparatus according to the invention is particularly well suited to use by a first responder, such as a doctor of the emergency ambulance service, a fire fighter, a nurse or the like, to ventilate an individual, namely a patient, in cardiopulmonary arrest, while that individual is being subjected to chest compressions (CCs) in the context of cardiac massage being performed by the said first responder.

1. A respiratory assistance apparatus (1) comprising:
a gas source (4) able to deliver a flow of respiratory gas in a ventilation circuit (2) intended to be connected to a patient (20), the flow of respiratory gas being delivered by the gas source (4) in such a way as to generate and maintain several gas pressure levels (Ph, Pb) for times of preset duration (Dh, Db),
a measurement device (6) able and designed to measure at least one flow parameter representative of the gas flow and chosen from the pressure of the gas and the gas flow rate, and to deliver at least one flow signal representative of the flow parameter,
a signal processing device (5) designed and able to:
i) analyse and deduce from the said flow signal, the existence or absence of chest compressions (CCs) performed on the patient,
ii) determine the value of the frequency (F) of the chest compressions (CCs) in the event that chest compressions (CCs) exist,
iii) supply the frequency value (F) to a display (7),
and the display (7) allowing information to be displayed,
wherein the signal processing device is designed to supply the display with a corrected frequency value (F_{c}) corresponding to the frequency value (F) determined immediately before the start of a time of duration (Dh, Db) considered, when the signal processing device does not detect a chest compression (CC) for part of the duration (Dh, Db) considered.

2. The apparatus of claim 1, wherein the display (7) is configured to display the corrected frequency value (F_{c}) for at least part of the duration (Dh, Db) considered.

3. The apparatus of claim 1, wherein the display (7) is configured to display, directly or indirectly, the frequency in the form of a numerical value, of a graphical representation, of an indication or of any illustrative symbol.

4. The apparatus of claim 1, wherein the gas source (4) delivers the flow of respiratory gas and maintains the gas pressure levels (Ph, Pb) of the flow, for times (Dh, Db) of preset duration such that:
the gas pressure level is kept at a high pressure (Ph) for a first duration (Dh), and
the gas pressure level is kept at a low pressure (Pb) for a second duration (Db), where:
0<Pb<Ph, the second duration (Db) following on from the first duration (Dh).

5. The apparatus of claim 1, further comprising a control system operating the gas source (4) in such a way as to deliver the flow of gas and to generate the pressure levels (Ph, Pb) for the preset durations (Dh, Db).

6. The apparatus of claim 5, wherein the gas source (4) is a motorized microblower.

7. The apparatus of claim 1, wherein the measurement device (6) comprises a flow sensor or a pressure sensor.

8. The apparatus of claim 1, wherein the signal processing device (5) comprises at least one microprocessor (8) implementing at least one algorithm.

9. The apparatus of claim 1, wherein the display (7) comprises a display screen.

10. The apparatus of claim 1 further comprising a filtering system configured to filter the flow signal representative of the flow parameter coming from the measurement device.

11. The apparatus of claim 1, further comprising a memory storage device (12) designed to store data.

12. The apparatus of claim 1, wherein the signal processing device (5) is configured to determine the value of the frequency (F) of the chest compressions (CCs) by calculating, over a given duration, the ratio between the number of chest compressions (CCs) and the given duration.

13. The apparatus of claim 1, designed for and capable of of counting the chest compressions (CCs) detected over a given period of time.

14. The apparatus of claim 1, wherein the signal processing device (5) is configured to supply the display (7) with the corrected frequency value (F_{c}), when the signal processing device (5) does not detect a chest compression (CC) over a time (Th, Tb) equal to 10 to 60% of the duration (Dh, Db) considered.
